(19) United States
(12) Patent Application Publication
(10) Pub. No.: US 2015/0038779 A1
(43) Pub. Date: Feb. 5, 2015

(54) Support Apparatus for Urinary Implant Cuff Valve

(71) Applicant: Philip Sacks, Laguna Woods, CA (US)

(72) Inventor: Philip Sacks, Laguna Woods, CA (US)

(21) Appl. No.: 13/954,813

(22) Filed: Jul. 30, 2013

(51) Int. Cl. A61F 2/00

(52) U.S. Cl. CPC A61F 2/00
USPC 600/37

(57) ABSTRACT

A Support Apparatus for Urinary Implant Cuff Valve. The device should be worn directly against the skin, underneath the underclothing. It has a pair of juxtaposed sling staps that pass beneath the wearer's genitals when the device is worn. These sling straps relieve pressure from the cuff valve when the wearer repositions from sitting to standing, and vice versa. A pair of side pads interconnect the sling straps with leg straps that are worn to encircle the upper thigh area of the wearer. An alternate version has an additional adjustable waist band that supports the legs straps by a plurality of vertical support straps.

### BACKGROUND OF THE INVENTION

1. Field of the Invention

This invention relates generally to therapeutic support garments and, more specifically, to a Support Apparatus for Urinary Implant Cuff Valve.

2. Description of Related Art

An artificial urinary sphincter (also known as “spinster” or AUS) involves a simple surgical procedure to help relieve lingering incontinence in men following prostate cancer treatment. The procedure can be done on an outpatient basis or with an overnight hospital stay. FIG. 1 depicts the essential elements of a prior art AUS

The urinary control system implant 10 comprises a cuff valve 12 encircling the urethra 14. The cuff valve 12 is made of a silicone rubber material. The surgical process consists of a doctor placing a small balloon 22 in the lower abdomen and a actuator pump 20 in the scrotum, in addition to the cuff valve 12. When the cuff 12 accumulates fluid, it will impose compressive force onto the urethra 14 so that urine will not be released. Patients simply squeeze the pump 20 a few times when they want to urinate. Once the pump 20 is activated the fluid flows from the cuff valve 12 to the balloon 22 via return tubing 24. The cuff valve 12 is designed to remain open for three to five minutes so that all of the urine can drain from the bladder 16. Once urination is completed, all fluid will flow back into the cuff valve 12, and will thereby block of the urethra 14 once again.¹ ¹ http://www.prostate-cancer.com/coping-with-prostate-cancer/incontinence-urinary-sphincters.html

While the AUS can be extremely liberating when successful at controlling urine flow, it can also become very problematic when it allows unwanted leakage. It has been noticed that one common instigator for leakage is when the patient stands up after having been seated for more than a few minutes. It is believed that this type of leakage is caused because remaining seated for prolonged periods can inadvertantly cause the fluid to be squeezed out of the pump 20 or cuff valve 12, and into the pressure regulating ballon 22. While seated, enough pressure is exerted on the urethra 14 to prevent leakage. However, when the patient stands up this pressure on the cuff valve 12 from being seated is removed, and urine can be allowed to leak by until the actuating fluid returns to the cuff valve 12 from the balloon 22.

The only known solutions to the AUS leakage problem are to add some sort of cushion or support device to the patient's chair, such as an inflatable ring. Carrying this sort of cushion is very inconvenient and may not be compatible with certain types of chairs or seats. What is needed is a device that is always on the patient's person to prevent inadvertant cuff leakage no matter where the patient wishes to sit down.

### SUMMARY OF THE INVENTION

In light of the aforementioned problems associated with the prior devices, it is an object of the present invention to provide a Support Apparatus for Urinary Implant Cuff Valve. The device should be worn directly against the skin, underneath the underclothing. It should have a pair of juxtaposed sling staps that pass beneath the wearer's genitals when the device is worn. These sling straps should relieve pressure from the cuff valve when the wearer repositions from sitting to standing, and vice versa. A pair of side pads should interconnect the sling straps with leg straps that are worn to encircle the upper thigh area of the wearer. An alternate version should be provided that has an additional adjustable waist band that supports the legs straps by a plurality of vertical support straps.

### BRIEF DESCRIPTION OF THE DRAWINGS

The objects and features of the present invention, which are believed to be novel, are set forth with particularity in the appended claims. The present invention, both as to its organization and manner of operation, together with further objects and advantages, may best be understood by reference to the following description, taken in connection with the accompanying drawings, of which:

FIG. 1 is a cutaway side view of an in-situ artificial urinary control system implant;

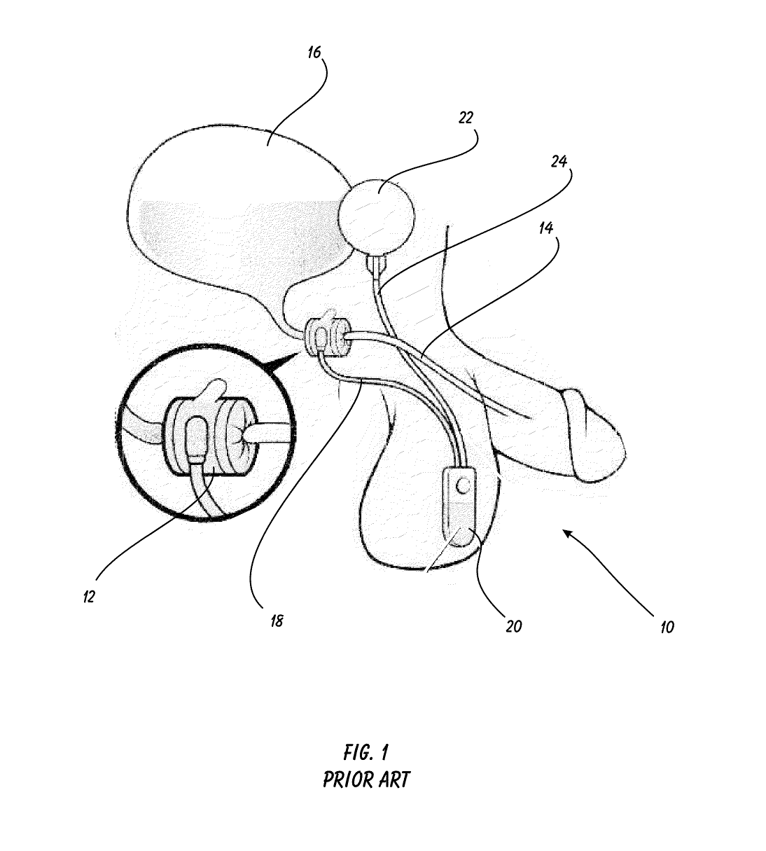

FIG. 2 is a front perspective view of a preferred embodiment of the cuff valve support apparatus of the present invention;

FIGS. 3A-3C are top, front and partial side views, respectively, of the apparatus of FIG. 2;

FIG. 4 is a perspective view of the apparatus of FIGS. 2 and 3 when donned by a user; and

FIG. 5 is a perspective view of an alternate design of the apparatus of FIGS. 2-4 when donned by a user.

### DETAILED DESCRIPTION OF THE PREFERRED EMBODIMENTS

The following description is provided to enable any person skilled in the art to make and use the invention and sets forth the best modes contemplated by the inventor of carrying out his invention. Various modifications, however, will remain readily apparent to those skilled in the art, since the generic principles of the present invention have been defined herein specifically to provide a Support Apparatus for Urinary Implant Cuff Valve.

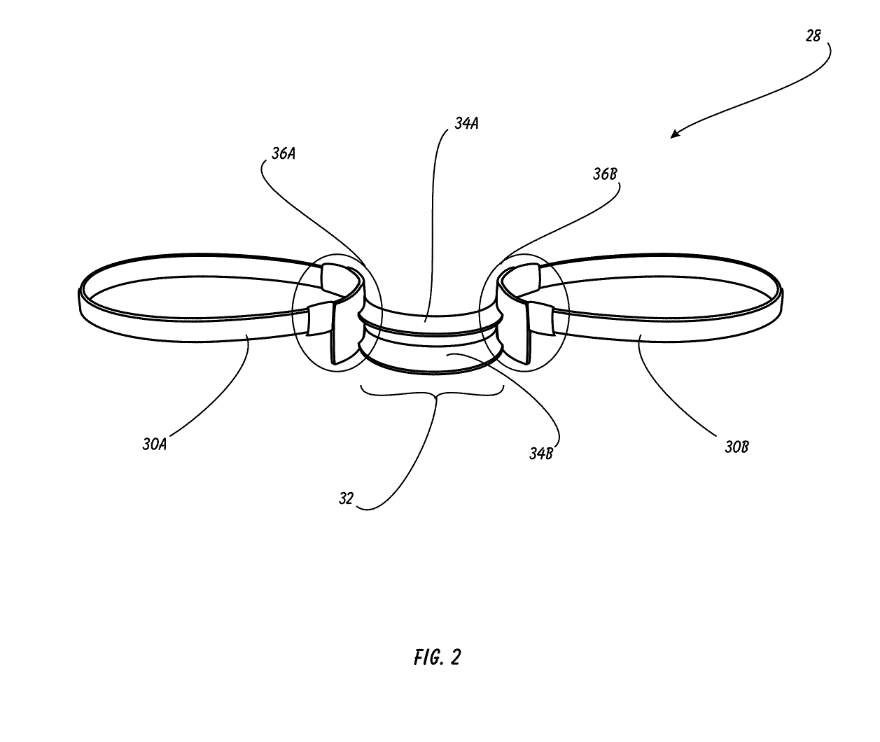

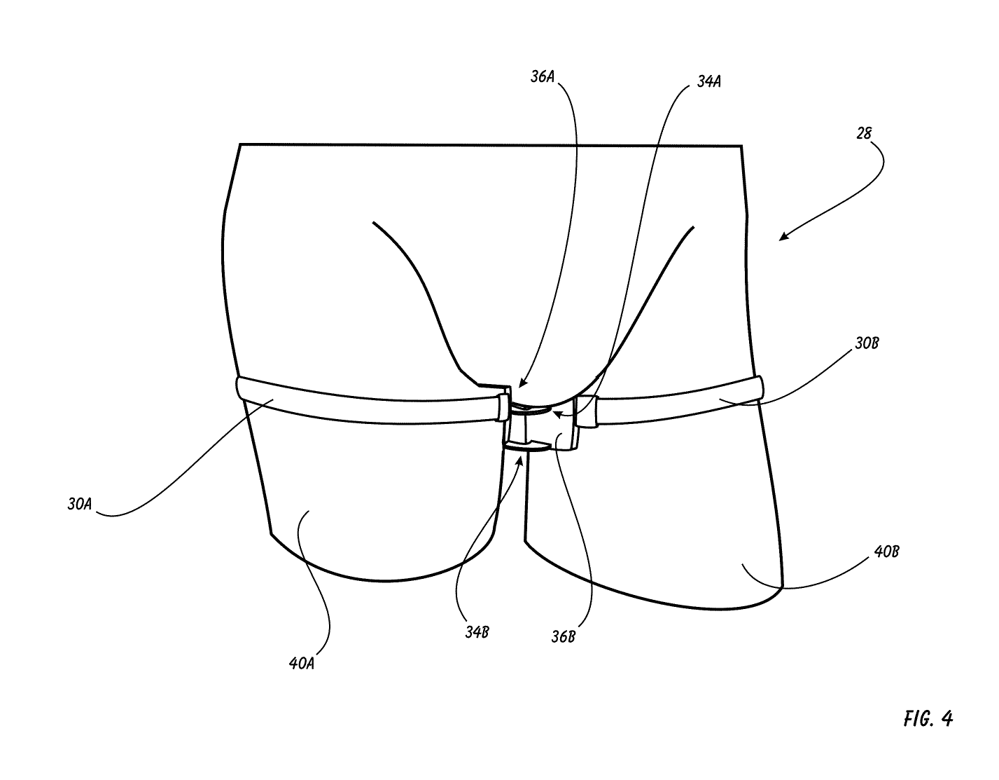

The present invention can best be understood by initial consideration of FIG. 2.² FIG. 2 is a front perspective view of a preferred embodiment of the cuff valve support apparatus 28 of the present invention. As is depicted below in FIG. 4, the apparatus 28 is designed to be worn by the user in the upper thigh area of both legs. A pair of leg straps 30A, 30B encircle the user's upper thighs. The straps 30A, 30B could be made from elasticised material (or have a cloth-covered elastic core), or could be adjustible in diameter and be provided with buckles, hook-and-loop fasteners, or other fastening means to adjust their diameter to provide a snug fit. ² As used throughout this disclosure, element numbers enclosed in square brackets [ ] indicates that the referenced element is not shown in the instant drawing figure, but rather is displayed elsewhere in another drawing figure.

First and second side pads 36A, 36B are attached to the facing sides of the two straps 30A, 30B. They are located as such so that they will provide cushioning to the user's upper thigh and genitals area when the apparatus 28 is being worn. A support sling 32 of one or more straps interconnects the two pads 36A, 36B. In the depicted version, there is an upper sling strap 34A and a lower sling strap 34B. These straps 34A, 34B are preferably made from elastic material so that they will provide strength, yet flexibility in their support.

As should be apparent, when the apparatus 28 is worn by the user in its proper location at the upper thighs, the support sling 32 will pass directly beneath the user's genitals. The upper support strap 34A is positioned to gently support the genitals, particularly when the user sits down. The lower strap 34B is positioned lower down, so as to guard the genital area from anything pressing against the genitals (and particularly against the cuff valve [12] or actuator pump [20]).

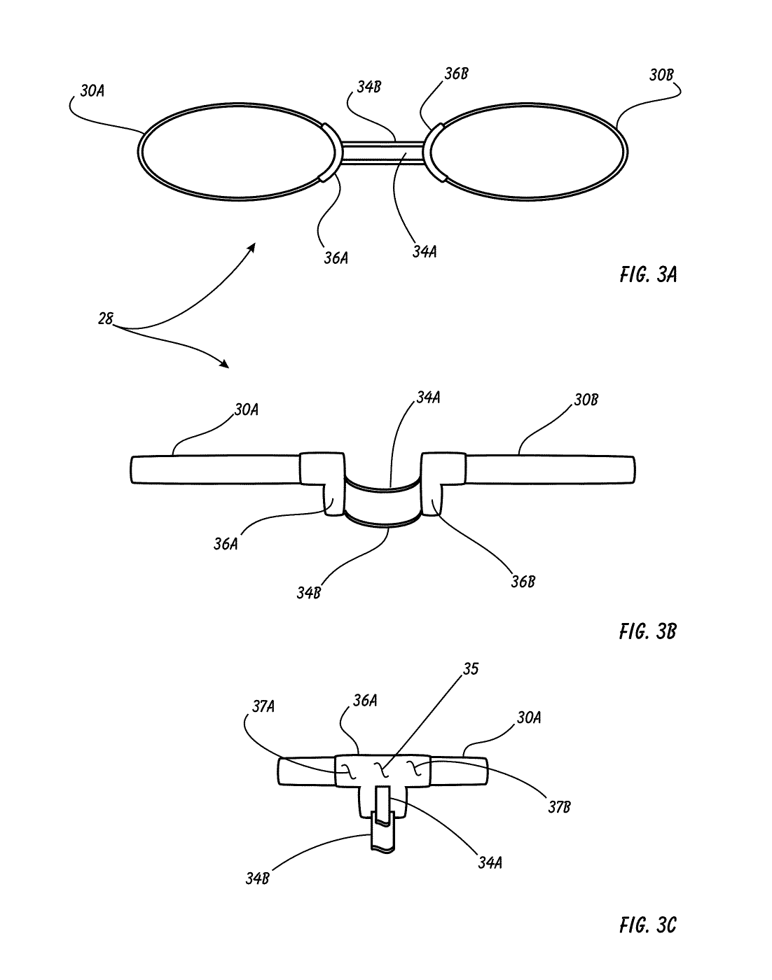

While it is not specifically known whether the common problem of cuff [12] leakage after being seated is caused by compression of the cuff [12], or by overpressure in one of the tubes [18] or [24], extensive testing has determined that the dual-level support sling 32 shown here has effectively eliminated this phenomena. It is believed that the upper sling strap 34A raises and supports the urethra [14] and potentially the cuff valve [12] so that pressure from the seating surface does not cause the cuff valve [12] to partially release its compression against the urethra [14]. As an added safety factor, the lower sling strap 34B prevents the user's clothing or other external elements from pressing against the components of the urinary control system implant [10]. FIGS. 3A-3C provide additional detail.

FIGS. 3A-3C are top, front and partial side views, respectively, of the apparatus 28 of FIG. 2. Here, we see that the lower sling strap 34B is marginally wider than the upper sling strap 34A. There is no specific ratio that has proven to be the most desireable between the widths, however, a narrower upper sling strap 34A tends to create less discomfort to the user, while the wider lower sling strap 34B provides an additional barrier without creating additional user discomfort.

FIG. 3C shows that the pads 36A, 36B are generally “T”-shaped in the instant design. The center portion 35 is larger and extends downwardly from the leg strap 30A by at least one inch (or more) to provide additional comfort for the user, as well as to create a mounting location for the lower sling strap 34B. The two end portions 37A, 37B extend toward the front and back of the leg strap 30A (when being worn), and are much narrower—essentially only covering the strap 30A in the genital area of the wearer.

The entire apparatus 28 is made from durable, washable materials. It is typically worn directly against the skin (i.e. underneath the undergarments), if desired. FIG. 4 depicts the device 28 as if worn beneath the undergarments.

FIG. 4 is a perspective view of the apparatus 28 of FIGS. 2 and 3 when donned by a user. As discussed above, the thigh straps 30A, 30B are worn at the upper thighs 40A, 40B so that the upper sling strap 34A is gently supporting the user's genitals. The lower sling strap 34B is then in spaced relation with the upper sling strap 34A. As can be seen, the first and second side pads 36A, 36B reside between the legs 40A, 40B and the genitals in order to stabilize them and to add comfort.

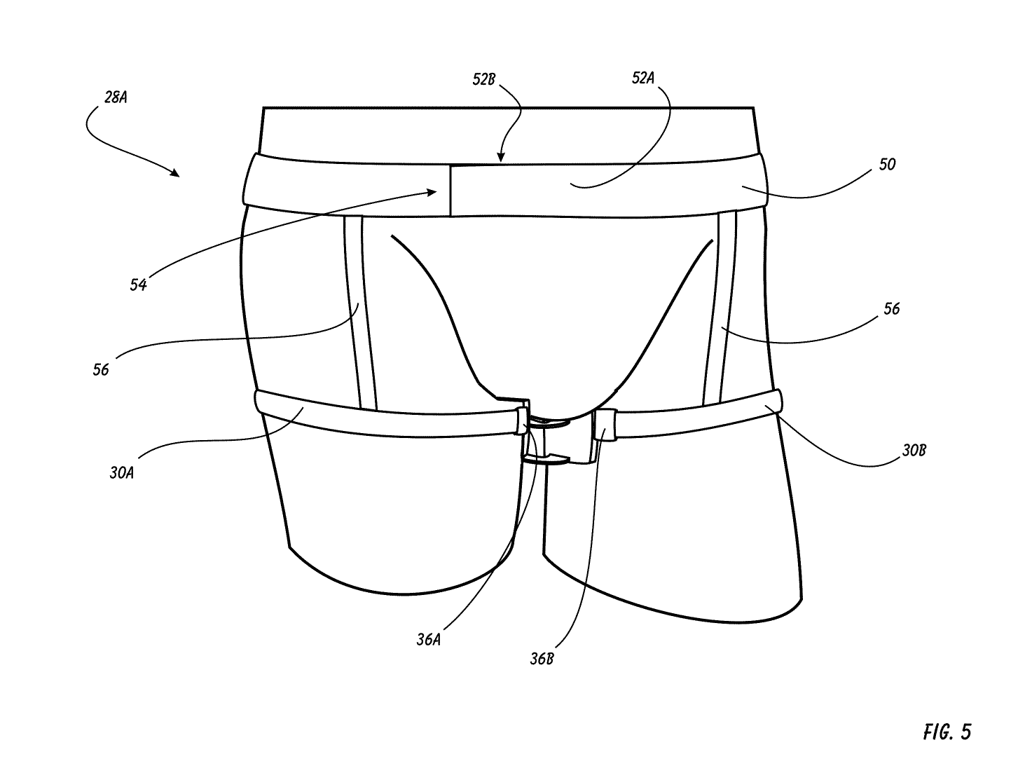

In the event that the wearer desires to have additional vertical support for the device to prevent slippage, an alternate version has been provided, and is depicted in FIG. 5. FIG. 5 is a perspective view of an alternate design 28A of the apparatus of FIGS. 2-4 when donned by a user. Here, a waist band 50 has been incorporated into the device 28A. The waist band 50 is preferably made from stronger, more durable, elastic material than is the rest of the device 28A. This is feasible since the waist band 50 is located around the user's torso or hips, rather than between (or around) the legs. The waist 50 terminates in first and second ends 52A, 52B. The waist band 50 has fastener pads 54 at the ends 52A, 52B so that the diameter can be adjusted and then fixed, so that the band 50 will fit properly. Two or more vertical support straps 56 interconnect the waist band 50 and the leg straps 30A, 30B (more straps 56 could be at the back-side of the device 28A—not depicted here).

Those skilled in the art will appreciate that various adaptations and modifications of the just-described preferred embodiment can be configured without departing from the scope and spirit of the invention. Therefore, it is to be understood that, within the scope of the appended claims, the invention may be practiced other than as specifically described herein.

What is claimed is:

1. A wearable support device, comprising:
a first leg strap;
a second leg strap; and
a support sling interconnecting said first and second leg straps.

2. The device of claim 1, further comprising:
a first side pad attached to said first leg strap and a first end of said support sling; and
a second side pad attached to said second leg strap and a second end of said support sling.

3. The device of claim 2, wherein said support sling comprises:
an upper support strap interconnecting said first and second side pads; and
a lower support strap interconnecting said first and second side pads in spaced relation to said upper support strap.

4. The device of claim 3, wherein said side pads comprise a center portion attached to said leg strap and extending down therefrom by one inch or more.

5. The device of claim 4, wherein said side pads further comprise end portions extending from opposite sides of said center portion and along a said leg strap, said end portions defining a width that is less than the width of said center portion.

6. The device of claim 5, wherein said support straps are made from elastic material.

7. A support and protection apparatus for a urethra cuff valve implant, comprising:
a right leg strap sized to encircle a user's upper right thigh;
a left leg strap sized to encircle a user's upper left thigh; and
a support sling interconnecting said right and left leg straps.

8. The device of claim 7, further comprising:
a first side pad attached to said right leg strap and a first end of said support sling; and
a second side pad attached to said left leg strap and a second end of said support sling.

9. The device of claim 8, wherein said support sling comprises:
an upper support strap interconnecting said first and second side pads; and
a lower support strap interconnecting said first and second side pads in spaced relation to said upper support strap.

10. The device of claim 9, wherein said side pads comprise a center portion attached to said leg strap and extending down therefrom by one inch or more.

11. The device of claim 10, wherein said side pads further comprise end portions extending from opposite sides of said center portion and along a said leg strap, said end portions defining a width that is less than the width of said center portion.

12. The device of claim 11, wherein said support straps are made from elastic material.

13. A method for preventing leakage from a patient's urethra cuff valve implant, said cuff implanted within the patient's genital area, the method comprising the steps of:
donning a support and protection apparatus, said apparatus comprising:
a right leg strap sized to encircle a user's upper right thigh;
a left leg strap sized to encircle a user's upper left thigh; and
a upper sling strap interconnecting said right and left leg straps; and
positioning said support and protection apparatus so that said right and left leg straps are encircling the upper thighs of the wearer and said upper sling strap is in contact with the patient's genitals.

14. The method of claim 13, wherein said apparatus of said donning and positioning steps further comprises
a first side pad attached to said right leg strap and a first end of said upper sling strap; and
a second side pad attached to said left leg strap and a second end of said upper sling strap.

15. The method of claim 14, wherein said apparatus of said donning and positioning steps further comprises a lower support strap interconnecting said first and second side pads in spaced relation to said upper support strap.

16. The method of claim 15, wherein said side pads of said apparatus of said donning and positioning steps further comprise a center portion attached to said leg strap and extending down therefrom by one inch or more.

17. The method of claim 16, wherein said side pads of said apparatus of said donning and positioning steps further comprise end portions extending from opposite sides of said center portion and along a said leg strap, said end portions defining a width that is less than the width of said center portion.

18. The method of claim 17, wherein said support straps of said apparatus of said donning and positioning steps are made from elastic material.

19. The device of claim 1, further including a waist strap parallel to, and in spaced relation to said leg straps, said waist strap attached to each said leg strap by one or more vertical support straps.

20. The apparatus of claim 7, further including a waist strap parallel to, and in spaced relation to said leg straps, said waist strap attached to each said leg strap by one or more vertical support straps.
